(19) United States
(12) Patent Application Publication
(10) Pub. No.: US 2009/0170898 A1
(43) Pub. Date: Jul. 2, 2009

(54) Sulphonamide Derivatives as Modulators of the Glucocorticoid Receptor

(76) Inventors: Malena Bengtsson, Lund (SE); Håkan Bladh, Lund (SE); Thomas Hansson, Lund (SE); Elizabeth Kinchin, Leicestershire (GB)

Correspondence Address: FISH & RICHARDSON P.C., P.O BOX 1022, MINNEAPOLIS, MN, 55440-1022, US

(21) Appl. No.: 12/295,092

(22) PCT Filed: Mar. 29, 2007
(86) PCT No.: PCT/SE07/00304

§ 371 Date: Sep. 29, 2008

Related U.S. Application Data
(60) Provisional application No. 60/788,274, filed on Mar. 31, 2006.

(51) Int. Cl. A61K 31/4709; C07D 401/10; C07D 403/14; A61K 31/416

(52) U.S. Cl. 514/314; 546/171; 548/361.1; 514/406

(57) ABSTRACT

Compounds of formula (I):

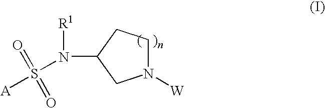

or a pharmaceutically acceptable salt thereof; compositions comprising them, processes for preparing them and their use in medical therapy (for example modulating the glucocorticoid receptor in a warm blooded animal).

The present invention relates to sulphonamide derivatives, to their use as medicaments (for example in the treatment of an inflammatory disease state), to pharmaceutical compositions comprising them and to processes for preparing them.

Sulphonamide derivatives are disclosed as anti-inflammatories in WO 2004/019935 and WO 2004/050631. Pharmaceutically active sulphonamides are also disclosed in Arch. Pharm. (1980) 313 166-173, J. Med. Chem. (2003) 46 64-73, J. Med. Chem. (1997) 40 996-1004, EP 0031954, EP 1190710 (WO 200124786), U.S. Pat. No. 5,861,401, U.S. Pat. No. 4,948,809, U.S. Pat. No. 3,992,441 to and WO 99/33786.

It is known that certain non-steroidal compounds interact with the glucocorticoid receptor (GR) and, as a result of this interaction, produce a suppression of inflammation (see, for example, U.S. Pat. No. 6,323,199). Such compounds can show a clear dissociation between anti-inflammatory and metabolic actions making them superior to earlier reported steroidal and non-steroidal glucocorticoids. The present invention provides further non-steroidal compounds as modulators (for example agonists, antagonists, partial agonists or partial antagonists) of the glucocorticoid receptor capable of having a dissociation between their anti-inflammatory and metabolic actions.

The present invention provides a compound of formula (I):

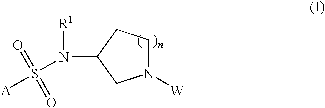

wherein:
n is 1 or 2;
A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy, phenyl, benzyl, benzyloxy, pyridinyl, pyridinyloxy or pyrazolyl; the substituents phenoxy, phenyl, benzyl, benzyloxy, pyridinyl, pyridinyloxy and pyrazolyl being optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹²R¹³;
R¹ is hydrogen, C₁₋₆ alkyl, phenyl, pyridinylC(O), C₃₋₆ cycloalkyl, (C₃₋₆ cycloalkyl)CH₂ or C₃₋₄ alkenyl;
W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl;
W is optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy (itself optionally substituted by halogen, C₁₋₄ alkyl, C₁₋₄ haloalkyl, C₁₋₄ alkoxy or C₁₋₄ haloalkoxy), imidazolyl, phenyl {itself optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NH₂, NH(C₁₋₄ alkyl) or N(C₁₋₄ alkyl)₂}, pyridyl {itself optionally substituted by halo, C₁₋₆ allyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NH₂, NH(C₁₋₄ alkyl) or N(C₁₋₄ alkyl)₂}, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹⁴R¹⁵;
R¹⁰, R¹¹, R¹², R¹³, R¹⁴ and R¹⁵ are independently, hydrogen, C₁₋₄ alkyl or C₃₋₇ cycloalkyl;
or a pharmaceutically acceptable salt thereof.

Compounds of formula (I) can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate, p-toluenesulphonate, succinate, glutarate or malonate.

The compounds of formula (I) may exist as solvates (such as hydrates) and the present invention covers all such solvates.

Halogen is, for example, fluorine or chlorine.

Alkyl groups and moieties are straight or branched chain and are, for example, methyl, ethyl, n-propyl, iso-propyl, n-butyl, sec-butyl or tert-butyl.

Haloalkyl (for example fluoroalkyl) comprises, for example, 1 to 6, such as 1, 2, 3, 4 or 5 halogen (for example fluorine) atoms. In one aspect it is, for example, CHF₂, CF₃, CH₂CF₃ or C₂F₅. In another aspect it is, for example, CHF₂, CF₃, CH₂CF₃, C₂F₅ or CH₂Cl. Haloalkoxy (for example fluoroalkoxy) comprises, for example, 1 to 6, such as 1, 2, 3, 4 or 5 halogen (for example fluorine) atoms. In one aspect it is, for example, OCHF₂, OCF₃, OCH₂CF₃ or OC₂F₅. In another aspect it is, for example, OCHF₂, OCF₃, OCH₂CF₃, OC₂F₅ or OCH₂Cl.

Cycloalkyl is for example, cyclopropyl, cyclopentyl or cyclohexyl.

In one particular aspect the present invention provides a compound of formula (I), wherein A is phenyl, pyridyl or pyrazolyl, each being optionally substituted by halogen, C₁₋₄ alkyl, C₁₋₄ haloalkyl, C₁₋₄ alkoxy, C₁₋₄ haloalkoxy, C₃₋₆ cycloalkyl or phenyl (itself optionally substituted by halogen, C₁₋₄ alkyl, C₁₋₄ haloalkyl, C₁₋₄ alkoxy or C₁₋₄ haloalkoxy).

In a further aspect the present invention provides a compound of formula (I), wherein A is phenyl optionally substituted by halogen (such as chloro or fluoro), C₁₋₄ alkyl (such as methyl), C₁₋₄ haloalkyl (such as CHF₂ or CF₃), C₁₋₄ alkoxy (such as methoxy) or C₁₋₄ haloalkoxy (such as OCF₃).

In another aspect the present invention provides a compound of formula (I), wherein A is pyridyl optionally substituted by halogen (such as chloro or fluoro), C₁₋₄ alkyl (such as methyl), C₁₋₄ haloalkyl (such as CHF₂ or CF₃), C₁₋₄ alkoxy (such as methoxy) or C₁₋₄ haloalkoxy (such as OCF₃).

In a still further aspect the present invention provides a compound of formula (I), wherein A is pyrazolyl optionally substituted by halogen (such as chloro or fluoro), C₁₋₄ alkyl (such as methyl), C₁₋₄ haloalkyl (such as CHF₂ or CF₃), C₁₋₄ alkoxy (such as methoxy), C₁₋₄ haloalkoxy (such as OCF₃), C₃₋₆ cycloalkyl or phenyl (itself optionally substituted by halogen, C₁₋₄ alkyl, C₁₋₄ haloalkyl, C₁₋₄ alkoxy or C₁₋₄ haloalkoxy).

In another aspect the present invention provides a compound of formula (I), wherein n is 1.

In yet another aspect the present invention provides a compound of formula (I), wherein n is 2.

In a further aspect the present invention provides a compound of formula (I), wherein R¹ is hydrogen.

In a still further aspect the present invention provides a compound of formula (I) wherein W is phenyl, pyridyl, indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl).

In another aspect the present invention provides a compound of formula (I) wherein W is indazol-4-yl, indazol-5-yl, indazol-6-yl, indazol-7-yl or quinolin-5-yl. For example W is indazol-4-yl (such as 1-substituted indazol-4-yl) or quinolin-5-yl.

In a further aspect the present invention provides a compound of formula (I) wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃), pyridyl (itself optionally substituted by halogen, C₁₋₄ alkyl, CF₃, C₁₋₄ alkoxy or OCF₃) or C(O)NH₂.

In another aspect the present invention provides a compound of formula (I) wherein W is phenyl optionally substituted by halogen, C₁₋₄ alkyl, CF₃, C₁₋₄ alkoxy or OCF₃.

In yet another aspect the present invention provides a compound of formula (I) wherein W is pyridyl optionally substituted by halogen, C₁₋₄ alkyl, CF₃, C₁₋₄ alkoxy or OCF₃.

In a still further aspect the present invention provides a compound of formula (I) wherein W is indazolyl (for example indazol-4-yl, indazol-5-yl, indazol-6-yl or indazol-7-yl) and it is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃), pyridyl (itself optionally substituted by halogen, C₁₋₄ alkyl, CF₃, C₁₋₄ alkoxy or OCF₃) or C(O)NH₂.

In another aspect the present invention provides a compound of formula (I) wherein W is indazolyl (for example indazol-4-yl, indazol-5-yl, indazol-6-yl or indazol-7-yl) substituted by phenyl (itself optionally substituted by halogen, C₁₋₄ alkyl, CF₃, C₁₋₄ alkoxy or OCF₃) or pyridyl (itself optionally substituted by halogen, C₁₋₄ alkyl, CF₃, C₁₋₄ alkoxy or OCF₃).

In yet another aspect the present invention provides a compound of formula (I) wherein W is indazolyl (for example indazol-4-yl) substituted by phenyl (itself optionally substituted by halogen (such as fluoro), C₁₋₄ alkyl (such as methyl), CF₃, C₁₋₄ alkoxy (such as methoxy) or OCF₃).

In a further aspect the present invention provides a compound of formula (I) wherein W is indazolyl (for example indazol-4-yl) substituted by pyridyl (itself optionally substituted by halogen (such as fluoro), C₁₋₄ alkyl (such as methyl), CF₃, C₁₋₄ alkoxy (such as methoxy) or OCF₃).

In another aspect the present invention provides a compound of formula (I) wherein n is 1 or 2; R¹ is hydrogen; A is phenyl (optionally substituted by halo, C₁₋₄ alkyl, C₁₋₄ alkoxy, C₁₋₄ haloalkyl or OCF₃) or pyrazolyl (optionally substituted by halo, C₁₋₄ alkyl, C₁₋₄ haloalkyl, C₃₋₆ cycloalkyl or phenyl); and, W is quinolinyl (for example quinolin-5-yl) or indazolyl (for example 1-substituted indazol-4-yl) optionally substituted by halophenyl (such as 4-fluorophenyl) or halopyridinyl (such as 2-fluoropyridin-5-yl).

The compounds of formula (I) can be prepared using or adapting methods disclosed in the art, or by using or adapting the method disclosed in the Examples below. Starting materials for the preparative methods are either commercially available or can be prepared by literature methods, adapting literature methods.

For example, a compound of the invention can be prepared by coupling a compound of formula (II):

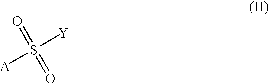

wherein Y is a leaving group (for example chlorine), with a compound of formula (III):

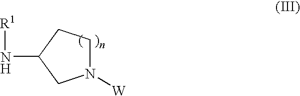

in a suitable solvent (such as tetrahydrofuran or N,N-dimethylformamide) at a temperature in the range −10° C. to 50° C.

The invention further provides processes for the preparation of the compounds of formula (I).

Because of their ability to bind to the glucocorticoid receptor the compounds of formula (I) are useful as anti-inflammatory agents, and can also display antiallergic, immunosuppressive and anti-proliferative actions. Thus, a compound of formula (I), or a pharmaceutically acceptable salt thereof can be used as a medicament for the treatment or prophylaxis of one or more of the following pathologic conditions (disease states) in a mammal (such as a human):
(i) Lung diseases, which coincide with inflammatory, allergic and/or proliferative processes:
   chronically obstructive lung diseases of any origin, mainly bronchial asthma
   bronchitis of different origins
   all forms of restructive lung diseases, mainly allergic alveolitis
   all forms of pulmonary edema, mainly toxic pulmonary edema
   sarcoidoses and granulomatoses, such as Boeck's disease
(ii) Rheumatic diseases/auto-immune diseases/degenerative joint diseases, which coincide with inflammatory, allergic and/or proliferative processes:
   all forms of rheumatic diseases, especially rheumatoid arthritis, acute rheumatic fever, polymyalgia rheumatica, collagenoses
   reactive arthritis
   inflammatory soft-tissue diseases of other origins
   arthritic symptoms in degenerative joint diseases (arthroses)
   traumatic arthritides
   collagen diseases of other origins, for example systemic lupus erythematodes, sclerodermia, polymyositis, dermatomyositis, polyarteritis nodosa, temporal arteritis
   Sjögren's syndrome, Still syndrome, Felty's syndrome
(iii) Allergies, which coincide with inflammatory, allergic and/or proliferative processes:
   All forms of allergic reactions, for example Quincke's edema, hay fever, insect bites, allergic reactions to pharmaceutical agents, blood derivatives, contrast media, etc., anaphylactic shock, urticaria, contact dermatitis
(iv) Dermatological diseases, which coincide with inflammatory, allergic and/or proliferative processes:
   atopic dermatitis (mainly in children)
   psoriasis
   erythematous diseases, triggered by different noxae, for example radiation, chemicals, burns, etc.
   acid burns
   bullous dermatoses
   diseases of the lichenoid group
   itching (for example of allergic origins)
   seborrheal eczema
   rosacea
   pemphigus vulgaris
   erythema exudativum multiforme
   erythema nodosum
   balanitis
   vulvitis
   inflammatory hair loss, such as alopecia areata
   cutaneous T-cell lymphoma
(v) Nephropathies, which coincide with inflammatory, allergic and/or proliferative processes:
   nephrotic syndrome
   all nephritides
(vi) Liver diseases, which coincide with inflammatory, allergic and/or proliferative processes:
   acute liver cell decomposition
   acute hepatitis of different origins, for example virally-, toxically- or pharmaceutical agent-induced
   chronically aggressive and/or chronically intermittent hepatitis
(vii) Gastrointestinal diseases, which coincide with inflammatory, allergic and/or proliferative processes:
   regional enteritis (Crohn's disease)
   ulcerative colitis
   gastroenteritis of other origins, for example native sprue
(viii) Proctological diseases, which coincide with inflammatory, allergic and/or proliferative processes:
   anal eczema
   fissures
   haemorrhoids
   idiopathic proctitis
(ix) Eve diseases, which coincide with inflammatory, allergic and/or proliferative processes:
   allergic keratitis, uvenitis iritis
   conjunctivitis
   blepharitis
   optic neuritis
   chorioiditis
   sympathetic ophthalmia
(x) Diseases of the ear-nose-throat area, which coincide with inflammatory, allergic and/or proliferative processes:
   allergic rhinitis, hay fever
   otitis externa, for example caused by contact dermatitis, infection, etc.
   otitis media
(xi) Neurological diseases, which coincide with inflammatory, allergic and/or proliferative processes:
   cerebral edema, mainly tumor-induced cerebral edema
   multiple sclerosis
   acute encephalomyelitis
   different forms of convulsions, for example infantile nodding spasms
(xii) Blood diseases, which coincide with inflammatory, allergic and/or proliferative processes:
   acquired haemolytic anemia
   idiopathic thrombocytopenia
(xiii) Tumor diseases, which coincide with inflammatory, allergic and/or proliferative processes:
   acute lymphatic leukaemia
   malignant lymphoma
   lymphogranulomatoses
   lymphosarcoma
   extensive metastases, mainly in breast and prostate cancers
(xiv) Endocrine diseases, which coincide with inflammatory, allergic and/or proliferative processes:
   endocrine orbitopathy
   thyrotoxic crisis
   de Quervain's thyroiditis
   Hashimoto's thyroiditis
   hyperthyroidism
(xv) Transplants, which coincide with inflammatory, allergic and/or proliferative processes;
(xvi) Severe shock conditions, which coincide with inflammatory, allergic and/or proliferative processes, for example anaphylactic shock
(xvii) Substitution therapy, which coincides with inflammatory, allergic and/or proliferative processes, with:
   innate primary suprarenal insufficiency, for example congenital adrenogenital syndrome
   acquired primary suprarenal insufficiency, for example Addison's disease, autoimmune adrenalitis, meta-infective, tumors, metastases, etc.
   innate secondary suprarenal insufficiency, for example congenital hypopituitarism
   acquired secondary suprarenal insufficiency, for example meta-infective, tumors, etc.
(xviii) Emesis, which coincides with inflammatory, allergic and/or proliferative processes:
   for example in combination with a 5-HT₃-antagonist in cytostatic-agent-induced vomiting.

Without prejudice to the foregoing, the compounds of formula (I) can also be used to treat disorders such as: Conies Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, oesophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cinhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis Sweet's disease, type 1 reactive leprosy, capillary hemangiomas, lichen planus, erythema nodosum acne, hirsutism, toxic epiderrmal necrolysis, erythema multiform, cutaneous T-cell lymphoma, psychoses, cognitive disorders (such as memory disturbances) mood disorders (such as depression and bipolar disorder), anxiety disorders and personality disorders.

As used herein the term “congestive heart failure” (CHF) or “congestive heart disease” refers to a disease state of the cardiovascular system whereby the heart is unable to efficiently pump an adequate volume of blood to meet the requirements of the body's tissues and organ systems. Typically, CHF is characterized by left ventricular failure (systolic dysfunction) and fluid accumulation in the lungs, with the underlying cause being attributed to one or more heart or cardiovascular disease states including coronary artery disease, myocardial infarction, hypertension, diabetes, valvular heart disease, and cardiomyopathy. The term “diastolic congestive heart failure” refers to a state of CHF characterized by impairment in the ability of the heart to properly relax and fill with blood. Conversely, the term “systolic congestive heart failure” refers to a state of CHF characterized by impairment in the ability of the heart to properly contract and eject blood.

As will be appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of physiological disorders contemplates both acute events and chronic conditions. In an acute event, compound is administered at the onset of symptoms and discontinued when the symptoms disappear.

In another aspect the present invention provides the use of a compound or formula (I), or a pharmaceutically acceptable salt thereof, for use in therapy (such as a therapy described above).

In yet another aspect the present invention provides the use of a compound or formula (I), or a pharmaceutically acceptable salt thereof, in the manufacture of a medicament for use in the treatment of a glucocorticoid receptor mediated disease state (such as a disease state described above).

In a further aspect the invention provides the use of a compound of formula (I), or a pharmaceutically acceptable salt thereof, in the manufacture of a medicament for use in the treatment of an inflammatory (such as an arthritic) condition.

In a still further aspect the invention provides the use of a compound of formula (I), or a pharmaceutically acceptable salt thereof, in the manufacture of a medicament for use in the treatment of an asthmatic or dermatological condition.

In another aspect the invention provides the use of a compound of formula (I), or a pharmaceutically acceptable salt thereof, in the manufacture of a medicament for use in the treatment of COPD.

The present invention further provides a method of treating a glucocorticoid receptor mediated disease state in a mammal (such as man), which comprises administering to a mammal in need of such treatment an effective amount of a compound of formula (I), or a pharmaceutically acceptable salt thereof.

In order to use a compound of formula (I), or a pharmaceutically acceptable salt thereof, for the therapeutic treatment of a mammal, said active ingredient is normally formulated in accordance with standard pharmaceutical practice as a pharmaceutical composition.

Therefore in another aspect the present invention provides a pharmaceutical composition comprising a compound of formula (I), or a pharmaceutically acceptable salt thereof, (active ingredient) and a pharmaceutically acceptable adjuvant, diluent or carrier. In a further aspect the present invention provides a process for the preparation of said composition comprising mixing the active ingredient with a pharmaceutically acceptable adjuvant, diluent or carrier. Depending on the mode of administration, the pharmaceutical composition can comprise from 0.05 to 99% w (percent by weight), for example from 0.05 to 80% w, such as from 0.10 to 70% w (for example from 0.10 to 50% w), of active ingredient, all percentages by weight being based on total composition.

A pharmaceutical composition of the present invention can be administered in a standard manner for the disease condition that it is desired to treat, for example by topical (such as to the lung and/or airways or to the skin), oral, rectal or parenteral administration (such as intravenous or ir intra-articular). Thus, a the compound of formula (I), or a pharmaceutically acceptable salt thereof, may be formulated into the form of, for example, an aerosol, a powder (for example dry or dispersible), a tablet, a capsule, a syrup, a granule, an aqueous or oily solution or suspension, an (lipid) emulsion, a suppository, an ointment, a cream, drops, or a sterile injectable aqueous or oily solution or suspension.

A suitable pharmaceutical composition of this invention is one suitable for oral administration in unit dosage form, for example a tablet or capsule containing between 0.1 mg and 1 g of active ingredient.

In another aspect a pharmaceutical composition of the invention is one suitable for intravenous, subcutaneous, intraarticular or intramuscular injection.

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. Tablets may be enteric coated by conventional means, for example to provide a coating of cellulose acetate phthalate.

The invention further relates to combination therapies or compositions wherein a GR agonist of formula (I), or a pharmaceutically acceptable salt thereof, or a pharmaceutical composition comprising a GR agonist of formula (I), or a pharmaceutically acceptable salt thereof, is administered concurrently (possibly in the same composition) or sequentially with one or more agents for the treatment of any of the above disease states.

For example, for the treatment of rheumatoid arthritis, osteoarthritis, COPD, asthma or allergic rhinitis a GR agonist of the invention can be combined with one or more agents for the treatment of such a condition. Where such a combination is to be administered by inhalation, then the one or more agents is selected from the list comprising:
a PDE4 inhibitor including an inhibitor of the isoform PDE4D;
a selective β.sub2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a muscarinic receptor antagonist (for example a M1, M2 or M3 antagonist, such as a selective M3 antagonist) such as ipratropium bromide, tiotropium bromide, oxitropium bromide, pirenzepine, glycopyrrolate (such as a glycopyrronium bromide) or telenzepine;
a steroid (such as budesonide or fluticasone);
a modulator of chemokine receptor function (such as a CCP1 receptor antagonist); or,
an inhibitor of p38 kinase function.

In another aspect of the invention where such a combination is for the treatment of COPD, asthma or allergic rhinitis the GR agonist of formula (I), or a pharmaceutically acceptable salt thereof, can be administered by inhalation or by the oral route and this is in combination with a xanthine (such as aminophylline or theophylline) which can be administered by inhalation or by the oral route.

The following Examples illustrate the invention. The following abbreviations are used in the Examples:
THF tetrahydrofuran
TFA trifluoroacetic acid
DMSO dimethylsulfoxide
DMF N,N-dimethylformamide
TBAT N,N,N-tributylbutan-1-aminium difluoro(triphenyl)silicate
DIEA diisopropylethyl amine
NMP 1-Methyl-2-pyrrolidinone
BINAP (R)-(+)-2,2′-Bis(diphenylphosphino)-1,1′-binaphthyl
Pd₂(dba)₃ Tris(dibenzylideneacetone)dipalladium(0)
app approximately
sat saturated
aq aqueous

### General Methods

NMR spectra were recorded on a 300 MHz Varian Mercury VXR instrument or a 400 MHz Varian UnityINOVA spectrometer or 500 MHz Varian UnityINOVA spectrometer. The central peaks of chloroform-d (δ_{H} 7.27 ppm), or DMSO-d₆ (δ_{H} 2.50 ppm) were used as internal references. Low resolution mass spectra and accurate mass determination were recorded on a Hewlett-Packard 1100 LC-MS system equipped with APCI ionisation chamber or on an Agilent 1100 LC/MSD system with APCI ionisation. Unless stated otherwise, starting materials were commercially available. All solvents and commercial reagents were of laboratory grade and were used as received.

The following methods were used for LC/MS analysis

Method A: Instrument Agilent 1100; Column C₁₈ Waters Symmetry 2.1×30 mm 3.5 μm; Flow rate 0.7 ml/min; Mass APCI; UV-absorption was measured at 254 nm; Solvent A: water+0.1% TFA; Solvent B: acetonitrile+0.1% TFA; Gradient 5-95%/B 8 min, 95% B 2 min.
Method B: Instrument Agilent 1100; Column Kromasil C₁₈ 3×100 mm 5 μm; Flow rate 1.0 ml/min; UV-absorption was measured at 254 nm; Solvent A: water+0.1% TFA; Solvent B: acetonitrile+0.1% TFA; Gradient 10-100% B 20 min, 100% B 1 min.

### EXAMPLE 1

### 2,4,6-Trimethyl-N-[(3S)-1-quinolin-5-ylpyrrolidin-3-yl]benzenesulfonamide

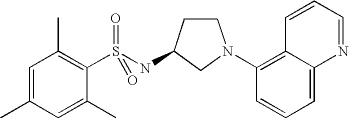

### 1a): tert-Butyl (3S)-1-quinolin-5-ylpyrrolidin-3-ylcarbamate

BINAP (0.032 mmole, 22 mg) and Pd₂(dba)₃ (0.011 mmole, 10 mg) were stirred in toluene(1 mL, 4 Å) under argon for 10 mins. (3S)-(−)-3-(tert-butoxycarbonylamino)-pyrrolidine (0.6 mmole, 111 mg) and 5-bromoquinoline (0.51 mmole, 105 mg) and then sodium tert-butoxide (0.71 mmole, 68 mg) were added. The reaction mixture was degassed and the reaction tube was filled with argon before it was heated in a microwave reactor (200W, 25 mins., 90° C.). Ethyl acetate was added and the mixture was filtered and evaporated. The product was purified on silica gel column chromatography (isohexane-ethyl acetate) to yield a yellow gum (103 mg).

APCI-MS m/z: 314.2 [MH+].

¹H NMR (399.99 MHz, CDCl₃) δ 8.88 (s, 1H), 8.50 (d, J=8.7 Hz, 1H), 7.72 (d, J=8.3 Hz, 1H), 7.59 (t, J=8.0 Hz, 1H), 7.35 (dd, J=8.6, 4.2 Hz, 1H), 6.97 (d, J=7.6 Hz, 1H), 4.91 (s, 1H), 4.41 (s, 1H), 3.64-3.53 (m, 2H), 3.31-3.20 (m, 2H), 2.47-2.36 (m, 1H), 1.98-1.87 (m, 1H), 1.48 (s, 9H)

### 1b): (3S)-1-Quinolin-5-ylpyrrolidin-3-amine

tert-Butyl (3S)-1-quinolin-5-ylpyrrolidin-3-ylcarbamate (103 mg) was stirred in TFA (5 ml) for 15 mins. at RT. The solvent was evaporated. 2M sodium hydroxide solution was added to the residue and the product was extracted into dichloromethane (3×20 ml). The organic layer was dried and evaporated to yield a yellow gum (60 mg).

APCI-MS m/z: 214.1 [MH+].

### 1c): 2,4,6-Trimethyl-N-[(3S)-1-quinolin-5-ylpyrrolidin-3-yl]benzenesulfonamide

(3S)-1-Quinolin-5-ylpyrrolidin-3-amine (60 mg, 0.28 mmole) and 2-mesitylenesulfonyl chloride (74 mg, 0.338 mmole) were dissolved in pyridine (2 mL) and stirred at room temperature overnight. The mixture was evaporated, dissolved in ethyl acetate (60 ml) and washed with water (3×10 ml) and brine (110 ml). The organic layer was dried, concentrated and purified by silica gel column chromatography (isohexane-ethyl acetate), then by RPHPLC-C₁₈, then by silica gel column chromatography (2% ammonia/methanol/dichloromethane) to yield a pale yellow solid (25 mg).

APCI-MS m/z: 396.0 [MH+].

¹H NMR (299.944 MHz, CDCl₃) δ 8.88 (dd, J=4.1, 1.7 Hz, 1H), 8.37 (ddd, J=8.6, 1.7, 0.9 Hz, 1H), 7.73 (d, J=8.4 Hz, 1H), 7.56 (t, J=8.1 Hz, 1H), 7.33 (dd, J=8.6, 4.2 Hz, 1H), 6.93 (s, 2H), 6.90 (dd, J=7.7, 0.9 Hz, 1H), 4.90 (d, J=8.1 Hz, 1H), 4.10-3.98 (m, 1H), 3.56-3.46 (m, 1H), 3.34 (dd, J=10.1, 5.5 Hz, 1H), 3.24-3.12 (m, 2H), 2.66 (s, 6H), 2.38-2.24 (m, 1H), 2.29 (s, 3H), 2.00-1.87 (m, 1H)

### EXAMPLE 2

### 2,4,6-Trichloro-N-[(3S)-1-quinolin-5-ylpyrrolidin-3-yl]benzenesulfonamide

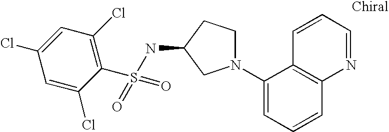

Was prepared analogous to Example 1c) but with corresponding starting material.

Purified by RPHPLC-C₁₈.

¹H NMR (499.881 MHz, DMSO-d₆) δ 8.94 (d, J=3.8 Hz, 1H), 8.84 (d, J=8.6 Hz, 1H), 8.62 (d, J=5.3 Hz, 1H), 7.70 (t, J=8.1 Hz, 1H), 7.59 (dd, J=8.6, 4.9 Hz, 1H), 7.54 (s, 2H), 7.42 (d, J=8.4 Hz, 1H), 6.86 (d, J=8.0 Hz, 1H), 4.05-3.98 (m, 1H), 3.68 (dd, J=10.4, 5.3 Hz, 1H), 3.59-3.50 (m, 1H), 3.50-3.33 (m, 2H obscured), 2.16-2.07 (m, 1H), 2.06-1.98 (m, 1H)

MS (APCI) e/z: 456.20 (MH)⁺

### EXAMPLE 3

### 2,4,6-Trichloro-N-[(3S)-1-[1-(6-fluoropyridin-3-yl)-1H-indazol-4-yl]pyrrolidin-3-yl]benzenesulfonamide

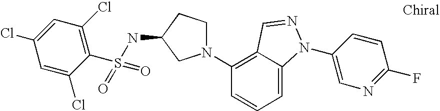

### 3a): 4-Bromo-1-(6-fluoropyridin-3-yl)-1H-indazole

4-Bromo-1H-indazole (600 mg) was dissolved in CH₂Cl₂ (28 mL). 2-Fluoropyridine-5-boronic acid (650 mg), anhydrous cupric acetate (620 mg) and pyridine (370 μL) were added followed by 4 Å molecular sieves (600 mg). The reaction mixture was stirred for 72 hours and then filtered through celite with methanol. The filtrate was evaporated and purified by silica gel column chromatography (isohexane-diethyl ether) to yield a pale orange solid (310 mg).

¹H NMR (399.99 MHz, CDCl₃) δ 8.62 (s, 1H), 8.28 (s, 1H), 8.20-8.14 (m, 1H), 7.61 (d, J=8.3 Hz, 1H), 7.45 (d, J=7.1 Hz, 1H), 7.35 (dt, J=0.2, 7.8 Hz, 1H), 7.16 (dd, J=8.8, 3.3 Hz, 1H)

MS (APCI) e/z: 292/293.9 (NH)+

### tert-Butyl (3S)-1-[1-(6-fluoropyridin-3-yl)-1H-indazol-4-yl]pyrrolidin-3-ylcarbamate

Was prepared analogous to Example 1a) but with corresponding starting material.

MS (APCI) e/z: 398.2 (MH)⁺

¹H-NMR (399.988 MHz, CDCl₃): δ 8.63 (s, 1H), 8.33 (s, 1H), 8.18-8.13 (m, 1H), 7.31 (t, J=8.1 Hz, 1H), 7.12 (dd, J=8.7, 3.2 Hz, 1H), 6.95 (d, J=8.1 Hz, 1H), 6.19 (d, J=7.8 Hz, 1H), 4.78 (s, 1H), 4.46 (s, 1H), 3.95 (dd, J=9.7, 5.8 Hz, 1H), 3.88-3.79 (m, 1H), 3.79-3.71 (m, 1H), 3.56-3.50 (m, 1H), 2.43-2.32 (m, 1H), 2.13-2.02 (m, 1H), 1.48 (s, 9H)

### (3S)-1-[1-(6-Fluoropyridin-3-yl)-1H-indazol-4-yl]pyrrolidin-3-amine

Was prepared analogous to Example 1b) but with corresponding starting material.

MS (APCI) e/z: 298.1 (MH)+

### 2,4,6-Trichloro-N-{(3S)-1-[1-(6-fluoropyridin-3-yl)-1H-indazol-4-yl]pyrrolidin-3-yl}benzenesulfonamide

Was prepared analogous to Example 1c) but with corresponding starting materials.

Purified by RPHPLC-C₁₈.

¹H NMR (499.881 MHz, DMSO-d₆) δ 8.60-8.56 (m, 2H), 8.31 (ddd, J=9.1, 6.7, 2.5 Hz, 1H), 8.24 (s, 1H), 7.57 (s, 2H), 7.37 (dd, J=8.8, 3.1 Hz, 1H), 7.19 (t, J=8.0 Hz, 1H), 6.91 (d, J=8.2 Hz, 1H), 6.04 (d, J=7.7 Hz, 1H), 4.05 (q, J=4.9 Hz, 1H), 3.70-3.60 (m, 2H), 3.50-3.25 (m, 2H obscured), 2.17-2.00 (m, 2H)

MS (APCI) e/z: 540.20 (MH)⁺

### EXAMPLE 4

### 2,4,6-Trichloro-N-{(3S)-1-[1-(4-fluorophenyl)-1H-indazol-4-yl]pyrrolidin-3-yl}benzenesulfonamide

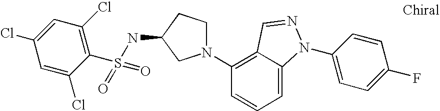

### tert-Butyl (3S)-1-[1-(4-fluorophenyl)-1H-indazol-4-yl]pyrrolidin-3-ylcarbamate

Was prepared analogous to Example 3a) and 1a) but with corresponding starting material.

MS (APCI) e/z: 397.2 (MH)⁺

¹H-NMR (299.944 MHz, CDCl₃): δ 8.29 (d, J=0.7 Hz, 1H), 7.71-7.63 (m, 2H), 7.30-7.17 (m, 3H), 6.95 (d, J=8.4 Hz, 1H), 6.16 (d, J=7.7 Hz, 1H), 4.78 (s, 1H), 4.45 (s, 1H), 3.98-3.89 (m, 1H), 3.89-3.69 (m, 2H), 3.53 (dd, J=9.9, 3.9 Hz, 1H), 2.43-2.29 (m, 1H), 2.13-2.00 (m, 1H), 1.48 (s, 9H)

### (3S)-1-[1-(4-Fluorophenyl)-1H-indazol-4-yl]pyrrolidin-3-amine

Was prepared analogous to Example 1b) but with corresponding starting material.

MS (APCI) e/z: 297.1 (MH)⁺

### 2,4,6-Trichloro-N-{(3S)-1-[1-(4-fluorophenyl)-1H-indazol-4-yl]pyrrolidin-3-yl}benzenesulfonamide

Was prepared analogous to Example 1c) but with corresponding starting material.

Purified by RPHPLC-C₁₈.

1H NMR (499.881 MHz, DMSO-d₆) δ 8.60 (d, J=5.8 Hz, 1H), 8.16 (s, 1H), 7.69 (dd, J=9.0, 4.8 Hz, 2H), 7.59 (s, 2H), 7.36 (t, J=8.8 Hz, 2H), 7.15 (t, J=8.1 Hz, 1H), 6.86 (d, J=8.4 Hz, 1H), 5.99 (d, J=7.7 Hz, 1H), 4.07-4.01 (m, 1H), 3.69-3.60 (m, 2H), 2.77-2.43 (m, 2H obscured), 2.15-1.99 (m, 2H)

MS (APCI) e/z: 539.15 (MH)⁺

### EXAMPLE 5

### 2,4-Dichloro-N-{(3S)-1-[1-(6-fluoropyridin-3-yl)-1H-indazol-4-yl]pyrrolidin-3-yl}-6-methylbenzenesulfonamide

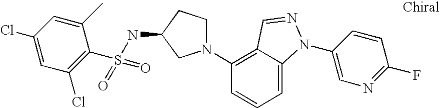

Was prepared analogous to Example 3) but with corresponding starting material.

Purified by RPHPLC-C₁₈.

¹H NMR (499.881 MHz, DMSO-d₆) δ 8.57 (d, J=2.0 Hz, 1H), 8.33-8.27 (m, 2H), 8.22 (s, 1H), 7.43 (d, J=2.2 Hz, 1H), 7.39-7.33 (m, 2H), 7.19 (t, J=8.1 Hz, 1H), 6.91 (d, J=8.2 Hz, 1H), 6.04 (d, J=7.7 Hz, 1H), 4.04-3.96 (m, 1H), 3.67-3.58 (m, 2H), 3.5-3.3 (m, 2H obscured), 2.55-2.45 (s, 3H obscured), 2.10-1.92 (m, 2H)

MS (APCI) e/z: 520.25 (MH)⁺

### EXAMPLE 6

### 2,4-Dichloro-6-methyl-N-[(3S)-1-quinolin-5-ylpyrrolidin-3-yl]benzenesulfonamide

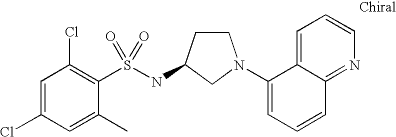

Was prepared analogous to Example 1c) but with corresponding starting material.

Purified by RPHPLC-C₁₈.

¹H NMR (499.881 MHz, DMSO-d₆) δ 8.93 (d, J=4.0 Hz, 1H), 8.81 (d, J=8.6 Hz, 1H), 8.32 (d, J=6.2 Hz, 1H), 7.70 (t, J=8.2 Hz, 1H), 7.57 (dd, J=8.6, 4.9 Hz, 1H), 7.42 (d, J=8.4 Hz, 1H), 7.38 (s, 1H), 7.29 (s, 1H), 6.86 (d, J=8.0 Hz, 1H), 3.97 (q, J=4.9 Hz, 1H), 3.62-3.56 (m, 1H), 3.5-3.4 (m, 1H obscured), 2.6-2.4 (m, 2H and s, 3H obscured), 2.15-1.91 (m, 2H)

MS (APCI) e/z: 436.25 (MH)⁺

### EXAMPLE 7

N-{(3S)-1-[1-(6-Fluoropyridin-3-yl)-1H-indazol-4-yl]pyrrolidin-3-yl}-2,4,6-trimethylbenzenesulfonamide

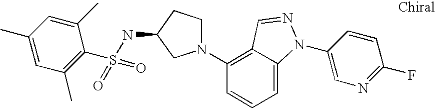

Was prepared analogous to Example 3 but with corresponding starting material.

Purified by RPHPLC-C₁₈.

¹H NMR (499.881 MHz, DMSO-d₆) δ 8.56 (d, J=2.7 Hz, 1H), 8.31-8.27 (m, 1H), 8.12 (s, 1H), 7.87 (d, J=6.9 Hz, 1H), 7.37 (dd, J=8.9, 2.7 Hz, 1H), 7.19 (t, J=7.8 Hz, 1H), 6.99 (s, 2H), 6.91 (d, J=8.9 Hz, 1H), 6.03 (d, J=7.9 Hz, 1H), 3.86 (q, J=8.8 Hz, 1H), 3.60-3.55 (m, 1H), 3.53-3.42 (m, 1H), 2.55-2.45 (s, 6H obscured), 2.22 (s, 3H), 2.06-1.97 (m, 2H), 1.94-1.84 (m, 2H)

MS (APCI) e/z: 480.35 (MH)⁺

### EXAMPLE 8

### N-{(3S)-1-[1-(4-Fluorophenyl)-1H-indazol-4-yl]pyrrolidin-3-yl}-2,4,6-trimethylbenzenesulfonamide

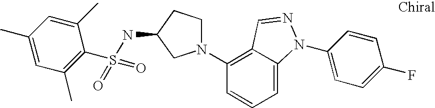

Was prepared analogous to Example 3 but with corresponding starting material.

Purified by RPHPLC-C₁₈.

¹H NMR (499.881 MHz, DMSO-d₆) δ 8.03 (s, 1H), 7.86 (d, J=7.1 Hz, 1H), 7.70-7.64 (m, 2H), 7.36 (t, J=14.9 Hz, 2H), 7.15 (t, J=8.0 Hz, 1H), 6.99 (s, 2H), 6.86 (d, J=8.2 Hz, 1H), 5.99 (d, J=8.0 Hz, 1H), 3.85 (q, J₁=6.1 Hz, 1H), 3.56 (q, J=7.4 Hz, 1H), 3.5-3.3 (m, 1H obscured), 2.55-2.45 (m, 2H and s, 6H obscured), 2.22 (s, 3H), 2.02 (sextet, J=6.4 Hz, 1H), 1.93-1.83 (m, 1H)

MS (APCI) e/z: 479.30 (MH)⁺

### EXAMPLE 9

### 1-Cyclopentyl-N-{(3S)-1-[1-(4-fluorophenyl)-1H-indazol-4-yl]pyrrolidin-3-Yl}-3,5-dimethyl-1H-pyrazole-4-sulfonamide

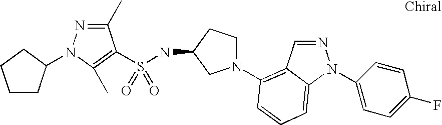

Was prepared analogous to Example 3 but with corresponding starting materials.

### 9a) 1-Cyclopentyl-3,5-dimethyl-1H-pyrazole-4-sulfonyl Chloride

2,4-Pentadion (5.5 g, 55 mmol), cyclopentylhydrazinhydrochloride (6.83 g, 50 mmol) and DIEA (9.58 ml, 55 mmol) were dissolved in ethanol and was refuxed for 48 hours. 0.5 M Citric acid solution and ethyl acetate were added and the organic phase was washed with NaHCO₃ and Brine. The organic layer was dried and evaporated to yield a colourless oil (6.70 g). The oil was dissolved in CHCl₃ (25 ml) chilled with ice and added to chloridosulfuric acid (30 ml). The mixture was stirred at 0° C. for one hour and was then refuxed for two hours. The mixture was allowed to reach room temperature, thionyl chloride (10 ml) was added and the mixture was refluxed for two hours. The solvent was evaporated and the residue was very slowly poured on ice/Na2CO3. Water was added to the chilled neutral solution and the resulting solid (11.4 g) was collected and dried.

MS (APCI) e/z: 263.75 (MH)⁺

### 1-Cyclopentyl-N-{(3S)-1-[1-(4-fluorophenyl)-1H-indazol-4-yl]pyrrolidin-3-yl}-3,5-dimethyl-1H-pyrazole-4-sulfonamide

Was prepared analogous to Example 3 but with corresponding starting materials

Purified by RPHPLC-C₁₈.

¹H NMR (499.881 MHz, DMSO-d₆) δ 8.18 (s, 1H), 7.73-7.62 (m, 3H), 7.40-7.33 (m, 2H), 7.17 (t, J=8.1 Hz, 1H), 6.87 (d, J=8.2 Hz, 1H), 6.01 (d, J=8.0 Hz, 1H), 4.63 (quintet, J=12.7 Hz, 1H), 3.80 (sextet, J=7.4 Hz, 1H), 3.62 (q, J=10.4 Hz, 1H), 3.57-3.48 (m, 1H), 2.76-2.56 (m, 1H), 2.55-2.45 (m, 1H and s, 3H obscured), 2.26 (s, 3H), 2.15-2.03 (m, 1H), 2.00-1.87 (m, 3H), 1.87-1.70 (m, 4H), 1.60-1.50 (m, 2H)

MS (APCI) e/z: 523.40 NH)⁺

### EXAMPLE 10

### 1-Cyclopentyl-3,5-dimethyl-N-[(3S)-1-quinolin-5-ylpyrrolidin-3-yl]-1H-pyrazole-4-sulfonamide

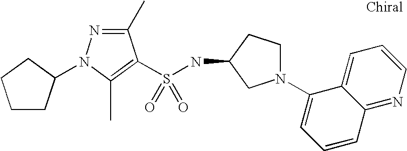

Was prepared analogous to Example 9 but with corresponding starting material.

Purified by RPHPLC-C₁₈

¹H NMR (499.881 MHz, DMSO-d₆) δ 8.93 (d, J=3.8 Hz, 1H), 8.86 (d, J=8.0 Hz, 1H), 7.74-7.67 (m, 2H), 7.59 (dd, J=8.6, 4.6 Hz, 1H), 7.45 (d, J=8.2 Hz, 1H), 6.89 (d, J=8.0 Hz, 1H), 4.60 (quintet, J=10.0 Hz, 1H), 3.79-3.71 (m, 1H), 3.56-3.21 (m, 2H obscured), 2.75-2.35 (m, 2H obscured), 2.38 (s, 3H), 2.21 (s, 3H), 2.11-2.03 (m, 1H), 1.94-1.81 (m, 3H), 1.79-1.67 (m, 4H), 1.59-1.48 (m, 2H)

MS (APCI) e/z: 440.30 (MH)⁺

### EXAMPLE 11

### 1-(Difluoromethyl)-N-{(3S)-1-[1-(4-fluorophenyl)-1H-indazol-4-yl]pyrrolidin-3-yl}-3,5-dimethyl-1H-pyrazole-4-sulfonamide

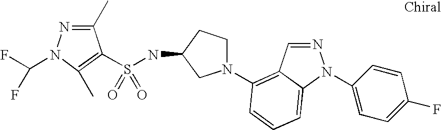

Was prepared analogous to Example 9) but with corresponding starting material.

Purified by RPHPLC-C₁₈.

¹H NMR (499.881 MHz, DMSO-d₆) δ 8.25 (s, 1H), 8.11 (d, J=6.6 Hz, 1H), 7.79 (s, 1H), 7.68 (dd, J=9.0, 5.0 Hz, 2H), 7.36 (t, J=8.5 Hz, 2H), 7.18 (t, J=8.1 Hz, 1H), 6.88 (d, J=8.2 Hz, 1H), 6.04 (d, J=7.7 Hz, 1H), 3.87 (q, J=5.7 Hz, 1H), 3.68-3.61 (m, 2H), 3.58-3.49 (m, 1H), 2.77-2.67 (m, 1H), 2.55-2.45 (s, 3H obscured), 2.32 (s, 3H), 2.15-2.05 (m, 1H), 1.97-1.86 (m, 1H).

MS (APCI) e/z: 505.25 (MH)⁺

### EXAMPLE 12

N-{(3S)-1-[1-(4-Fluorophenyl)-1H-indazol-4-yl]pyrrolidin-3-yl}-2,4-dimethylbenzenesulfonamide

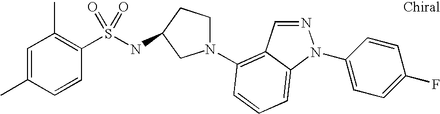

Was prepared analogous to Example 3 but with corresponding starting material.

Purified by RPHPLC-C₁₈.

¹H NMR (499.881 MHz, DMSO-d₆) δ 8.07 (s, 1H), 7.98 (d, J=6.2 Hz, 1H), 7.73 (d, J=13.2 Hz, 1H), 7.68-7.65 (m, 2H), 7.36 (t, J=15.8 Hz, 2H), 7.19-7.11 (m, 3H), 6.86 (d, J=19.5 Hz, 1H), 6.00 (d, J=7.7 Hz, 1H), 3.84-3.75 (m, 1H), 3.55-3.25 (m, 2H obscured), 2.75-2.4 (m, 2H, and s, 3H obscured), 2.28 (s, 3H), 2.08-1.96 (m, 1H), 1.95-1.82 (m., 1H)

MS (APCI) e/z: 465.35 (MH)⁺

### EXAMPLE 13

### 2,4-Dimethyl-N-[(3S)-1-quinolin-5-ylpyrrolidin-3-yl]benzenesulfonamide

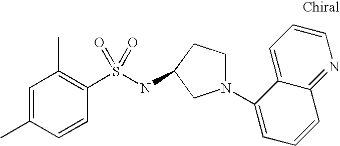

Was prepared analogous to Example 1c) but with corresponding starting material.

Purified by RPHPLC-C₁₈.

¹H NMR (499.881 MHz, DMSO-d₆) δ 8.87 (d, J=4.7 Hz, 1H), 8.69 (d, J=8.3 Hz, 1H), 7.97 (d, J=6.5 Hz, 1H), 7.65 (t, J=12.1 Hz, 2H), 7.53-7.49 (m, 1H), 7.43 (d, J=9.2 Hz, 1H), 7.03 (d, J=10.0 Hz, 2H), 6.85 (d, J=8.2 Hz, 1H), 3.80-3.70 (m, 1H), 3.24-3.17 (m, 2H), 2.78-2.66 (m, 1H), 2.55-2.45 (m, 1H and s, 3H obscured), 2.22 (s, 3H), 2.07-1.97 (m, 1H), 1.91-1.82 (m, 1H)

MS (APCI) e/z: 382.25 (MH)⁺

### EXAMPLE 14

### 1-Cyclopentyl-N-{(3S)-1-[1-(6-fluoropyridin-3-yl)-1H-indazol-4-yl]pyrrolidin-3-yl}-3,5-dimethyl-1H-pyrazole-4-sulfonamide

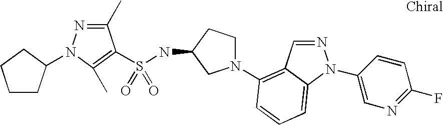

Was prepared analogous to Example 9 but with corresponding starting material.

Purified by RPHPLC-C₁₈.

¹H NMR (499.881 MHz, DMSO-d₆) δ 8.56 (s, 1H), 8.32-8.25 (m, 2H), 7.71 (d, J=6.9 Hz, 1H), 7.37 (dd, J=8.4, 3.0 Hz, 1H), 7.21 (t, J=8.0 Hz, 1H), 6.93 (d, J=8.2 Hz, 1H), 6.05 (d, J=8.1 Hz, 1H), 4.64 (quintet, J=7.3 Hz, 1H), 3.81 (sextet, J=7.3 Hz, 1H), 3.67-3.60 (m, 1H), 3.58-3.49 (m, 1H), 2.55-2.45 (s, 3H obscured), 2.25 (s, 3H), 2.13-2.03 (m, 2H), 2.01-1.89 (m, 4H), 1.88-1.70 (m, 4H), 1.61-1.51 (m, 2H)

MS (APCI) e/z: 524.30 (MH)⁺

### EXAMPLE 15

### N-{(3S)-1-[1-(4-Fluorophenyl)-1H-indazol-4-yl]pyrrolidin-3-yl}-5-methyl-1-phenyl-1H-pyrazole-4-sulfonamide

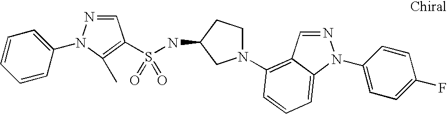

Was prepared analogous to Example 9 but with corresponding starting material.

Purified by RPHPLC-C₁₈.

¹H NMR (499.881 MHz, DMSO-d₆) δ 8.25 (s, 1H), 8.00 (d, J=12.5 Hz, 1H), 7.95 (s, 1H), 7.67 (dd, J=9.1, 5.1 Hz, 2H), 7.50 (dd, J=15.7, 6.6 Hz, 5H), 7.37 (t, J=8.8 Hz, 2H), 7.18 (t, J=7.9 Hz, 1H), 6.88 (d, J=7.7 Hz, 1H), 6.06 (d, J=8.0 Hz, 1H), 3.97-3.90 (m, 1H), 3.72-3.64 (m, 2H), 3.58-3.50 (m, 1H), 2.77-2.69 (m, 1H), 2.55-2.45 (s, 3H obscured), 2.21-2.09 (m, 1H), 2.04-1.93 (m, 1H)

MS (APCI) e/z: 517.25 (MH)⁺

### EXAMPLE 16

### 5-Methyl-1-phenyl-N-[(3S)-1-quinolin-5-ylpyrrolidin-3-yl]-1H-pyrazole-4-sulfonamide

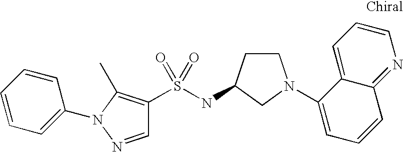

Was prepared analogous to Example 9 but with corresponding starting material.

Purified by RPHPLC-C₁₈.

¹H NMR (499.881 MHz, DMSO-d₆) δ 8.86 (d, J=5.0 Hz, 1H), 8.76 (d, J=8.1 Hz, 1H), 8.00 (d, J=6.5 Hz, 1H), 7.89 (s, 1H), 7.65 (t, J=8.3 Hz, 1H), 7.54-7.40 (m, 7H), 6.91 (d, J=8.0 Hz, 1H), 3.91-3.84 (m, 1H), 3.34-3.27 (m, 1H), 2.77-2.66 (m, 1H), 2.40 (s, 3H obscured), 2.20-2.09 (m, 2H), 1.96-1.85 (m, 2H).

MS (APCI) e/z: 434.30 (MH)⁺

### EXAMPLE 17

### 4-Bromo-N-[(3S)-1-quinolin-5-ylpyrrolidin-3-yl]-2-(trifluoromethoxy)-benzenesulfonamide

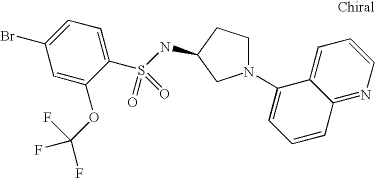

Was prepared analogous to Example 1c) but with corresponding starting material.

Purified by RPHPLC-C₁₈.

¹H NMR (499.881 MHz, DMSO-d₆) δ 8.92 (d, J=4.8 Hz, 1H), 8.82 (d, J=8.5 Hz, 1H), 8.47 (d, J=6.4 Hz, 1H), 7.82 (d, J=8.4 Hz, 1H), 7.72-7.67 (m, 3H), 7.58 (dd, J=8.8, 4.9 Hz, 1H), 7.46 (d, J=8.2 Hz, 1H), 6.90 (d, J=8.0 Hz, 1H), 3.95 (sextet, J=6.9 Hz, 1H), 3.55-3.35 (m, 1H obscured), 3.30 (dd, J=10.0, 4.4 Hz, 1H), 2.8-2.35 (m, 2H obscured), 2.15-2.06 (m, 1H), 1.95-1.87 (m, 1H)

MS (APCI) e/z: 516.20 (MH)⁺

### EXAMPLE 18

### 1-(Difluoromethyl)-3,5-dimethyl-N-[(3S)-1-quinolin-5-ylpyrrolidin-3-yl]-1H-pyrazole-4-sulfonamide

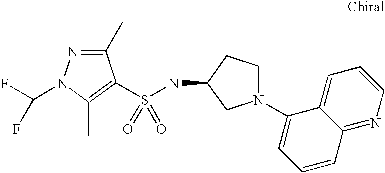

Was prepared analogous to Example 1c) but with corresponding starting material.

Purified by RPHPLC-C₁₈.

¹H NMR (499.881 MHz, DMSO-d₆) δ 8.93 (d, J=3.5 Hz, 1H), 8.87 (d, J=8.4 Hz, 1H), 8.13 (d, J=6.6 Hz, 1H), 7.75 (t, J=57.1 Hz, 1H), 7.70 (t, J=8.2 Hz, 1H), 7.59 (dd, J=8.6, 4.9 Hz, 1H), 7.46 (d, J=8.4 Hz, 1H), 6.92 (d, J=8.0 Hz, 1H), 3.83 (q, J=5.8 Hz, 1H), 3.58-3.50 (m, 1H), 3.55-3.45 (m, 1H obscured), 2.77-2.4 (m, 2H, and s, 3H obscured), 2.27 (s, 3H), 2.15-2.05 (m, 1H), 1.91-1.81 (m, 1H)

MS (APCI) e/z: 422.30 (MH)⁺

### EXAMPLE 19

### N-{(3S)-1-[1-(4-Fluorophenyl)-1H-indazol-4-yl]pyrrolidin-3-yl}-2,5-dimethylbenzenesulfonamide

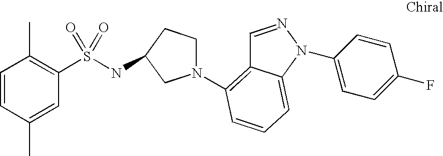

Was prepared analogous to Example 3 but with corresponding starting material.

Purified by RPHPLC-C₁₈.

¹H NMR (499.881 MHz, DMSO-d₆) δ 8.12 (s, 1H), 8.02 (d, J=7.8 Hz, 1H), 7.69-7.64 (m, 3H), 7.36 (t, J=8.8 Hz, 2H), 7.27 (d, J=8.8 Hz, 1H), 7.22 (d, J=7.8 Hz, 1H), 7.16 (t, J=8.3 Hz, 1H), 6.86 (d, J=8.1 Hz, 1H), 5.99 (d, J=7.8 Hz, 1H), 3.88-3.80 (m, 1H), 3.65-3.57 (m, 1H), 3.50-3.30 (m, 1H obscured), 2.80-2.30 (m, 2H obscured), 2.26 (s, 3H), 2.09-1.97 (m, 1H), 1.96-1.84 (m, 1H)

MS (APCI) e/z: 465.20 (MH)⁺

### EXAMPLE 20

### N-{(3S)-1-[1-(4-Fluorophenyl)-1H-indazol-4-yl]piperidin-3-yl}-2,4,6-trimethylbenzenesulfonamide

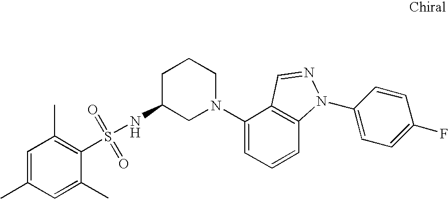

### tert-Butyl (3S)-3-[(mesitylsulfonyl)amino]piperidine-1-carboxylate

Was prepared analogous to Example 1c) but with corresponding starting material.

MS (APCI) e/z: 283.10 (M-BOC)⁺

¹H-NMR (399.99 MHz, CDCl₃): δ 6.97 (s, 2H), 4.67 (s, 1H), 3.60-2.97 (m, 5H), 2.66 (s, 6H), 2.31 (s, 3H), 1.79-1.38 (m, 4H), 1.44 (s, 9H)

### 2,4,6-Trimethyl-N-[(3S)-piperidin-3-yl]benzenesulfonamide

Was prepared analogous to Example 1b) but with corresponding starting material.

MS (APCI) e/z: 283.10 (MH)⁺

### N-{(3S)-1-[1-(4-Fluorophenyl)-1H-indazol-4-yl]piperidin-3-yl}-2,4,6-trimethylbenzenesulfonamide

Was prepared analogous to Example 1a) but with corresponding starting material. Purified by silica gel column chromatography (isohexane-ethyl acetate), then by RPHPLC-C₁₈.

MS (APCI) e/z: 493.20 (MH)⁺

¹H-NMR (399.99 MHz, DMSO-d₆): δ 7.88-7.83 (m, 2H), 7.74 (dd, J=9.0, 4.8 Hz, 2H), 7.43 (t, J=8.8 Hz, 2H), 7.30-7.19 (m, 2H), 7.04 (s, 2H), 6.43 (d, J=7.1 Hz, 1H), 3.47 (t, J=11.0 Hz, 2H), 3.29-3.17 (m, 1H), 2.78 (t, J=10.5 Hz, 1H), 2.70-2.61 (m, 1H), 2.60 (s, 6H), 2.27 (s, 3H), 1.85-1.71 (m, 2H), 1.66-1.52 (m, 1H), 1.48-1.34 (m, 1H)

### EXAMPLE 21

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 μL; assay buffer 10×, Stabilizing Peptide, DTT and ice cold MQ water) was added to the background wells. GS Red solution (7 μL; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nM and a dichroic mirror at 561 nm). The IC50 values were calculated using XLfit model 205 and are shown, for certain compounds of the invention, in the table below.

| | GRhuFL_FP_v2 (GR-binders) |
|---|---|
| Example No | IC50 (nM) |
| 1 | 48 |
| 2 | 17 |
| 3 | 19 |
| 4 | 25 |
| 5 | 25 |
| 6 | 26 |
| 7 | 38 |
| 8 | 40 |
| 9 | 120 |

1. A compound of formula (I): 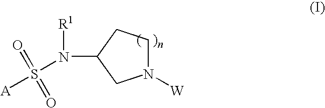
wherein:
n is 1 or 2;
A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ haloalkyl, C₁₋₄ haloalkoxy, C₃₋₆ cycloalkyl, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy, phenyl, benzyl, benzyloxy, pyridinyl, pyridinyloxy or pyrazolyl; the substituents phenoxy, phenyl, benzyl, benzyloxy, pyridinyl, pyridinyloxy and pyrazolyl being optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹²R¹³;
R¹ is hydrogen, C₁₋₆ alkyl, phenyl, pyridinylC(O), C₃₋₆ cycloalkyl, (C₃₋₆ cycloalkyl)CH₂ or C₃₋₄ alkenyl;
W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl;
W is optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy (itself optionally substituted by halogen, C₁₋₄ alkyl, C₁₋₄ haloalkyl, C₁₋₄ alkoxy or C₁₋₄ haloalkoxy), imidazolyl, phenyl {itself optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NH₂, NH(C₁₋₄ alkyl) or N(C₁₋₄ alkyl)₂}, pyridyl {itself optionally substituted by halo, C₁₋₆ alkyl, C₁₋₆ alkoxy, C₁₋₄ alkylthio, C₁₋₄ haloalkyl, C₁₋₄ haloalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NH₂, NH(C₁₋₄ alkyl) or N(C₁₋₄ alkyl)₂}, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹⁴R¹⁵;
R¹⁰, R¹¹, R¹², R¹³, R¹⁴ and R¹⁵ are independently, hydrogen, C₁₋₄ alkyl or C₃₋₇ cycloalkyl;
or a pharmaceutically acceptable salt thereof.

2. A compound of formula (I) as claimed in claim 1 wherein A is phenyl, pyridyl or pyrazolyl, each being optionally substituted by halogen, C₁₋₄ alkyl, C₁₋₄ haloalkyl, C₁₋₄ alkoxy, C₁₋₄ haloalkoxy, C₃₋₆ cycloalkyl or phenyl (itself optionally substituted by halogen, C₁₋₄ alkyl, C₁₋₄ haloalkyl, C₁₋₄ alkoxy or C₁₋₄ haloalkoxy).

3. A compound of formula (I) as claimed in claim 1 wherein n is 1.

4. A compound of formula (I) as claimed in claim 1 wherein R¹ is hydrogen.

5. A compound of formula (I) as claimed in claim 1 wherein W is phenyl, pyridyl, indolyl, indazolyl, quinolinyl or isoquinolinyl, each of which is optionally substituted as recited in claim 1.

6. A compound of formula (I) as claimed in claim 1 wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃), pyridyl (itself optionally substituted by halogen, C₁₋₄ alkyl, CF₃, C₁₋₄ alkoxy or OCF₃) or C(O)NH₂.

7. A compound of formula (I) as claimed in claim 1 wherein W is indazolyl and is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃), pyridyl (itself optionally substituted by halogen, C₁₋₄ alkyl, CF₃, C₁₋₄ alkoxy or OCF₃) or C(O)NH₂.

8. A process for preparing a compound o formula (I) as claimed in claim 1, the process comprising coupling a compound of formula (II): 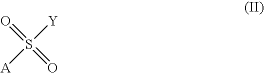
wherein Y is a leaving group (for example chlorine), with a compound of formula (III): 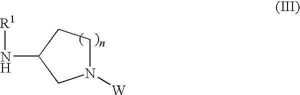
in a suitable solvent (such as tetrahydrofuran or N,N-dimethylformamide) at a temperature in the range −10° C. to 50° C.

9. A pharmaceutical composition comprising a compound or formula (I) as claimed in claim 1, or a pharmaceutically acceptable salt thereof, and a pharmaceutically acceptable adjuvant, diluent or carrier.

10-11. (canceled)

12. A method of treating a glucocorticoid receptor mediated disease state in a mammal, which comprises administering to a mammal in need of such treatment an effective amount of a compound of formula (I), or a pharmaceutically acceptable salt thereof as claimed in claim 1.

13. A combination of a compound of formula (I), or a pharmaceutically acceptable salt thereof, as claimed in claim 1, and one or more agents selected from the list comprising:
a PDE4 inhibitor;
a selective β.sub2. adrenoceptor agonist;
a muscarinic receptor antagonist;
a steroid;
a modulator of chemokine receptor function; or,
an inhibitor of p38 kinase function.
